(19) United States
(12) Patent Application Publication
(10) Pub. No.: US 2024/0197306 A1
(43) Pub. Date: Jun. 20, 2024

(54) PHOTOCHEMICAL TISSUE BONDING CLAMP AND IRRADIATION CHAMBER AND METHOD OF USE THEREOF

(71) Applicant: The General Hospital Corporation, Boston, MA (US)

(72) Inventor: Jonathan M. Winograd, Weston, MA (US)

(73) Assignee: The General Hospital Corporation, Boston, MA (US)

(21) Appl. No.: 18/555,407

(22) PCT Filed: Apr. 13, 2022
(86) PCT No.: PCT/US2022/024701

§ 371 Date: Oct. 13, 2023

Related U.S. Application Data
(60) Provisional application No. 63/174,482, filed on Apr. 13, 2021.

(51) Int. Cl. A61B 17/00; A61B 17/11; A61B 18/00; A61B 18/18

(52) U.S. Cl. CPC A61B 17/00491; A61B 17/1128; A61B 18/18; A61B 2017/1132; A61B 2018/0063

(57) ABSTRACT

A device, kit, and method for performing photochemical tissue bonding on a biological structure is provided. The device includes a first channel, a second channel, and an open window. The first channel is sized to receive a first portion of the biological structure, the second channel is sized to receive a second portion of the biological structure. The open window is positioned between the first channel and the second channel so that a third portion of the biological structure is exposed within the open window when the first channel receives the first portion and the second channel receives the second portion, where photochemical tissue bonding may be applied to the third portion.

### CROSS-REFERENCE TO RELATED APPLICATIONS

This application is based on, claims priority to, and incorporates herein by reference in its entirety, U.S. Provisional Patent Application Ser. No. 63/174,482 filed on Apr. 13, 2021.

### BACKGROUND

Clinical outcomes of nerve repair have not significantly improved over the last few decades, despite significant advances in the understanding of nerve regeneration. For example, standard suture repair is associated with various inherent disadvantages, such as nerve trauma resulting from needle passage, suture material invoking a foreign body response and inflammation, and suture tracks presenting a possible nidus for infection. In addition to these drawbacks, a suture attachment method of nerve repair does not seal the regenerating nerve environment. As a result, neurotrophic and neurotropic factors important for axon regeneration can leak from the nerve stumps at the site of repair. If not tightly sealed, axons can also escape from the nerve, reducing nerve regeneration and leading to painful neuroma formation. Also, alternative fixation methods such as fibrin glue or laser welding have been investigated but have not shown an improvement over microsurgery.

On the other hand, photochemical tissue bonding (PTB) can provide a water-tight method of sutureless repair for nerves and has been shown to enhance the quality and rapidity of neural regeneration when used at a neurorrhaphy site to seal the repair. In particular, PTB uses visible light to create covalent bonds between apposed tissue proteins that have been prestained with a nontoxic, photoactive dye (e.g., Rose Bengal). Dye photoactivation creates reactive oxygen species, cross-linking between amino acid residues, and the formation of non-thermal bonds. PTB has also been applied as a platform technology to a variety of other tissues such as, but not limited to, blood vessels.

Therefore, it would be desirable to provide systems and methods to accomplish photochemical tissue bonding of nerves, blood vessels, and/or other tissues.

### SUMMARY

In some aspects, a device for performing photochemical tissue bonding on a biological structure is provided. The device includes a first channel, a second channel, and an open window. The first channel is sized to receive a first portion of the biological structure, the second channel is sized to receive a second portion of the biological structure. The open window is positioned between the first channel and the second channel so that a third portion of the biological structure is exposed within the open window when the first channel receives the first portion and the second channel receives the second portion, where photochemical tissue bonding may be applied to the third portion.

In some aspects, a kit for performing photochemical tissue bonding on a biological structure is provided. The kit comprises a device with a first channel, a second channel, and an open window. The first channel is sized to receive a first portion of the biological structure, the second channel is sized to receive a second portion of the biological structure. The open window is positioned between the first channel and the second channel so that a third portion of the biological structure is exposed within the open window when the first channel receives the first portion and the second channel receives the second portion, where photochemical tissue bonding may be applied to the third portion. The kit further includes a photoactive dye.

In some aspects, a method for applying photochemical tissue bonding to a biological structure using a device comprising a first channel, a second channel, and an open window is provided. The method includes positioning the biological structure relative to the device, including positioning a portion of the biological structure in the open window, and preparing the biological structure for photochemical tissue bonding. The method further includes irradiating the portion of the biological structure within the window to accomplish photochemical tissue bonding, and releasing the biological structure from the device.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a side view of a two ends of a nerve to be repaired via photochemical tissue bonding.

FIG. 2 is diagram illustrating a method for nerve grafting via photochemical tissue bonding.

FIG. 3 is a side view of a two ends of a vessel repaired via photochemical tissue bonding.

FIG. 4 is a top view of a photochemical tissue bonding device according to some aspects.

FIG. 5 is a top view of two panels of the device of FIG. 4.

FIG. 6 is a partial top vie of a half-channel of one of the panels of FIG. 5.

FIG. 7 is a top view of the device of FIG. 4 including a nerve positioned therein, in according with some aspects.

FIG. 8 is a top view of the device of FIG. 4, including a nerve positioned therein, and a background material and wrap.

FIG. 9 is a partial top view of the device of FIG. 4, with the wrap partially wrapped around the nerve.

FIG. 10 is another partial top view of the device of FIG. 4, with the wrap fully wrapped around the nerve.

FIG. 11 is an isometric view of the device of FIG. 4, including a nerve positioned therein, and a light source.

FIG. 12 is a kit for photochemical tissue bonding of a biological structure, according to some aspects.

FIG. 13 is a method for photochemical tissue bonding, according to some aspects.

FIG. 13A is a first sub-method of the method of FIG. 13.

FIG. 13B is a second sub-method of the method of FIG. 13.

FIG. 13C is a third sub-method of the method of FIG. 13.

FIG. 13D is a fourth sub-method of the method of FIG. 13.

### DETAILED DESCRIPTION

Photochemical tissue bonding (PTB) can provide a water-tight method of sutureless repair for nerves and has been shown to enhance the quality and rapidity of neural regeneration when used at a neurorrhaphy site to seal the repair. In particular, PTB uses visible light to create covalent bonds between apposed tissue proteins that have been prestained with a nontoxic, photoactive dye (e.g., Rose Bengal). Dye photoactivation creates reactive oxygen species, cross-linking between amino acid residues, and the formation of non-thermal, water-tight bonds.

In recent years, such light-activated sealing of nerve repair sites with amnion nerve wraps has emerged as an alternative to standard suture, resulting in superior functional and histologic outcomes. These positive observations are likely attributable to a reduction in scar tissue formation, reduced axonal escape, and reduced leakage of neuro-regenerative factors. It is possible that growth-promoting and anti-fibrotic factors within amniotic membrane may also be involved. Furthermore, the absence of thermal damage using PTB provides an advantage over, for example, laser welding.

PTB has been applied to progressively more challenging nerve reconstructive scenarios, including direct repairs as well as large gap nerve injury models. Additionally, PTB has been applied employing nerve autografts as well as acellular nerve allografts, all with enhanced histologic and functional outcomes. For nerve grafting, the human amnion was strengthened and made more resistant to biodegradation with chemical cross-linking to withstand weakening over the longer periods of time necessary for neural regeneration. Autografts repaired with crosslinked human amnion (“xHAM”) and PTB (“xHAM/PTB”) in a large gap peripheral nerve injury study showed higher quality regeneration with improved functional measures, higher fiber diameters, and myelin thickness and improved muscle mass recovery compared with sutured autografts. Acellular nerve allografts in the same large gap model also showed significant improvements with the use of xHAM/PTB, with levels of recovery comparable to sutured autografts. In another study, a large (four-centimeter) nerve gap radial nerve injury model in a nonhuman primate compared sutured autografts to human acellular nerve allografts repaired with xHAM/PTB. Functional and electrophysiologic outcomes comparing the autograft and allograft, as in smaller animal models, were remarkably similar, demonstrating a significant improvement in the expected outcome for acellular nerve allografts attributable to the benefits of the PTB sealed neurorrhaphy.

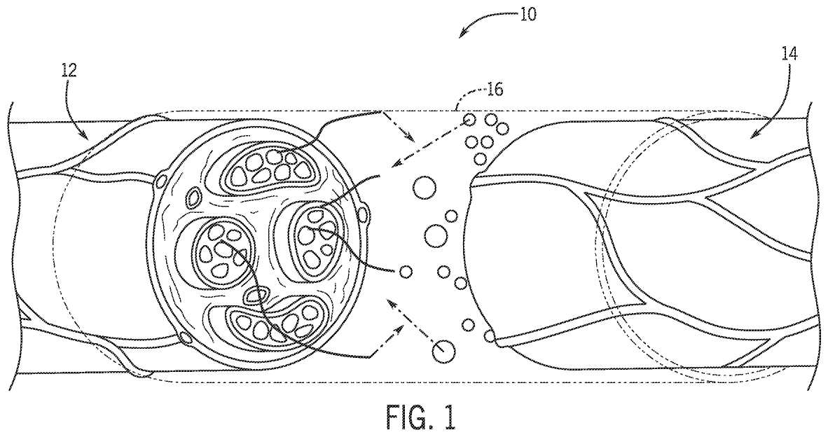

For example, FIG. 1 illustrates a repaired nerve 10 (e.g., an end-to-end repair) including a proximal nerve end 12, a distal nerve end 14, and a wrap 16. The wrap 16, such as an amnion nerve wrap, can be prestained with a photoactive dye and wrapped around the two nerve ends 12, 14. The wrap 16 can then be illuminated with light, such as a via laser or LED, at a certain wavelength or within a wavelength range to activate the photoactive dye. For example, when the photoactive dye is Rose Bengal, light at a wavelength 532 nanometers can activate the dye. Additionally, the wrap 16 can be crosslinked.

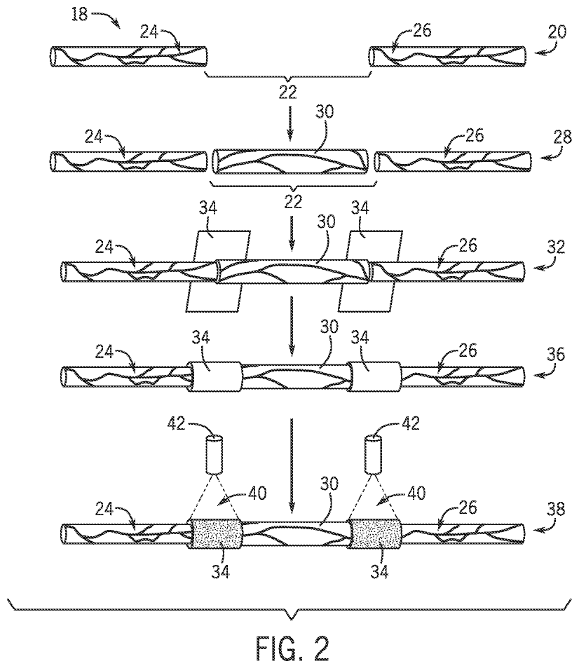

In another example, FIG. 2 illustrates a limb nerve repair method 18. First, step 20 illustrates a nerve gap 22 between nerve ends 24, 26. At step 28, an acellular human allograft 30 is inserted into the nerve gap 22. At step 32, crosslinked Rose Bengal-stained amnion wraps 34 are positioned at the nerve coaptation sites (e.g., the sites between nerve ends 24, 26 and the allograft 30). At step 36, the wraps 34 are circumferentially wrapped around respective sites. At step 38, the wraps 34 are illuminated (e.g., exposed to light 40 at a certain wavelength from a light source 42) to activate the Rose Bengal and accomplish PTB at the sites.

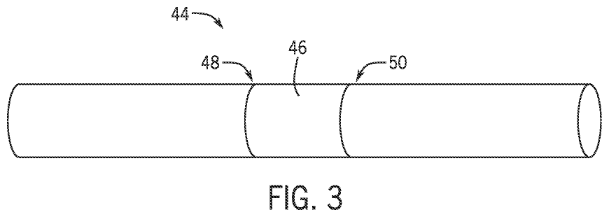

PTB has also been applied as a platform technology to a variety of other tissues such as, but not limited to, blood vessels and tendons. For example, in related studies, two ends of a blood vessel to be repaired were coated with Rose Bengal dye. A dissolvable bioglass stent was placed within the vessels to ensure good contact between the surfaces of the two lumens, and the area of the repair was then irradiated with green light to accomplish anastomosis. The stent then dissolved, leaving an intact patent vessel repair. In such studies, it was shown that PTB with a stent produced strong water-tight microvascular anastomoses. PTB also provided significant time saving, a de-skilled procedure, reduced inflammatory response, and reduced intimal hyperplasia compared to standard suture repair. FIG. 3 illustrates an example repaired vessel 44, including a bond site 46 where two vessel ends 48, 50 were joined (e.g., with an internal bioglass stent, not shown).

In yet another example, PTB repair can be used in place of traditional sutures for tendon transection repair. For example, an electrospun silk wrap, prestained with photoactive dye, can be wrapped over the tissue ends and illuminated, causing PTB.

According to the above examples, PTB involves applying a photoactive dye (e.g., Rose Bengal), apposing tissues, and illuminating the dye with light at a specific wavelength (e.g., green light when using Rose Bengal). According to some embodiments, the disclosure provides a system, kit, and/or method for accomplishing this PTB process for repairing nerves, vessels, tendons, or other channels or tissues of the body. More specifically, the system, kit, and method of some aspects are intended to facilitate the process of photochemical tissue bonding of a nerve, blood vessel or any solid or cannulated biological structures of a tubular nature, in clinical practice, by stabilizing the biological structure and stabilizing any wrap material around the structure to maximize the bonding area and facilitate the irradiation of the tissues and/or the wrap, which have been dyed with a photoactive dye.

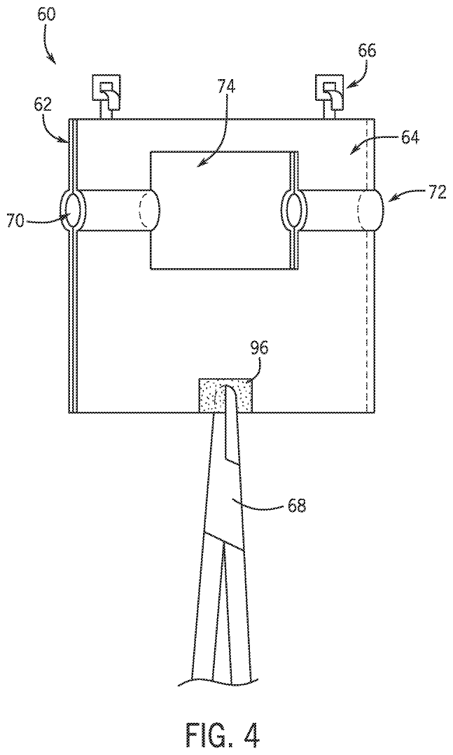

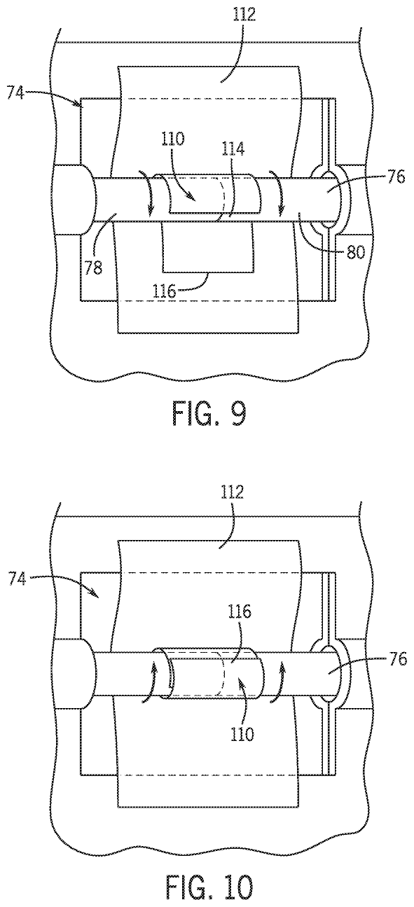

FIG. 4 illustrates a device 60 according to some aspects. For example, the device 60 can be a two-piece hinged clamp including a first panel 62, a second panel 64, and a hinge 66. In some aspects, the device 60 can be fixed in a clamped position by a clamping tool such as, but not limited to, a hemostat 68. Additionally, the device 60 can define a first channel 70, a second channel 72, and a window 74. Generally, a first end 78 of a biological structure 76 (e.g., a nerve, vessel, etc., such as the nerve illustrated in FIGS. 7-11) can be positioned within the first channel 70 and exposed within the window 74, and a second end 80 of the biological structure 76 can be positioned within the second channel 72 and exposed within the window 74. The first and second ends 78, 80, by being exposed within the window 74, can be directly exposed to a light source (not shown in FIG. 4) to effect photochemical tissue bonding.

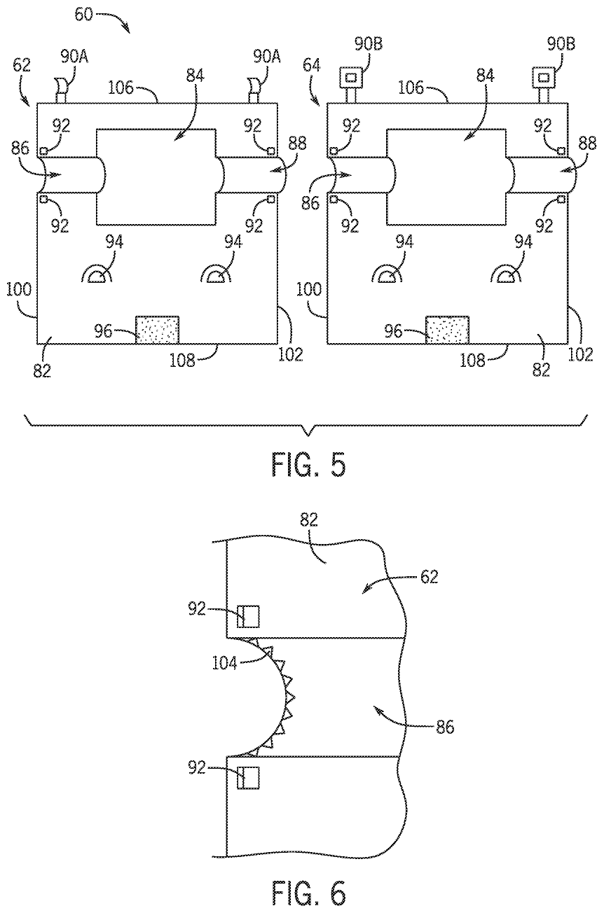

FIG. 5 illustrates the first and second panels 62, 64 separated from each other. The first panel 62 can include a base 82, a cutout 84 extending through the base 82, a first half-channel 86 (e.g., a hemicylindrical channel) positioned adjacent a first side of the cutout 84, a second half-channel 88 (e.g., a hemicylindrical channel) positioned adjacent a second side of the cutout 84, one or more first hinge portions 90A, suture holes 92, attachment sites or “shoes” 94, and a clamping surface 96. The second panel 64 can generally include the same features as the first panel 62, e.g., a base 82, a cutout 84, a first half-channel 86, a second half-channel 88, suture holes 92, shoes 94, and a clamping surface 96, as well as one or more second hinge portions 90B configured to engage the first hinge portions 90A. In some aspects, each panel 62, 64 can include equal lengths and/or widths such that the panels 62, 64 completely overlap each other when in a closed or clamped state, as further described below. Additionally, in some aspects, each panel 62, 64 can include a relatively small thickness, thus providing an ultra-thin device profile.

Still referring to FIG. 5, generally, a biological structure 76 can first be positioned relative to the first panel 62. More specifically, as shown in FIG. 5, the first half-channel 86 can extend from a first side 100 of the panel 62 to the cutout 84, and the second half-channel 88 can extend from an opposite second side 102 of the panel 62 to the cutout 84. A first end 78 of the biological structure 76 can be positioned within the first half-channel 86 and extend into the cutout 84, and a second end 80 of a biological structure 76 can be positioned within the second half-channel 88 and extend into the cutout 84. As a result, the first and second ends 78, 80 are exposed within the cutout 84 apposing one another and can be in direct contact with one another.

Accordingly, the half-channels 86, 88 can be sized to accommodate the biological structure 76. For example, in some aspects, the half-channels 86, 88 can each include a diameter between about 2 millimeters (mm) and about 2 centimeters (cm) (e.g., including, but not limited to, about 2 mm, about 3 mm, about 4 mm, about 5 mm, about 6 mm, about 1 cm, about 1.5 cm and/or about 2 cm). In another example, the half-channels 86, 88 can each include a diameter sized to accommodate specific biological structures 76 having a diameter range, such as, but not limited to: digital nerves, typically about 1-2 mm in diameter; radial sensory nerves, about 3-4 mm in diameter; median and ulnar nerves, about 5-6 mm in diameter; and/or larger nerves sizes for injured and scarred nerves, between 7-10 mm and/or 11-15 mm in diameter. Furthermore, while the device 60 illustrated herein includes one first half-channel 86 and one second half-channel 88 of each panel 62, 64, in some aspects, each panel 62, 64 can include multiple first half-channels 86 and multiple second half-channels 88, each having a different diameter so that the device 60 can accommodate biological structures 76 of different sizes.

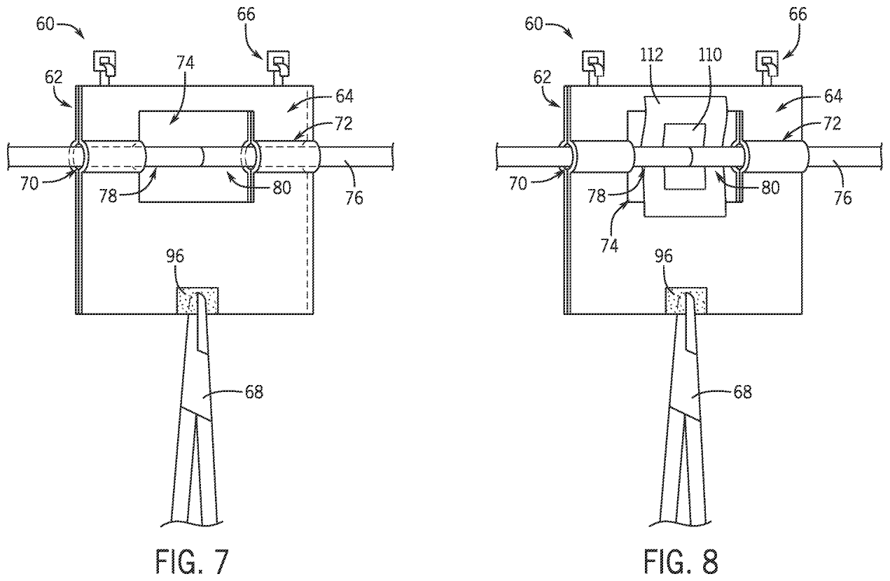

Thus, the half-channels 86, 88 can help stabilize the biological structures 76 for the bonding procedure, for example, by helping remove tension on the ends 78, 80. In some aspects, the biological structure 76 can be further stabilized through sutures (not shown) and/or end features on the half-channels 86, 88. More specifically, sutures (e.g., stay sutures) can be coupled to both the outer surface of the biological structure 76 and to an edge of the half-channel via the suture holes 92. Additionally or alternatively, the half-channels 86, 88 can include one or more end features such as, but not limited to, serrations 104, as shown in FIG. 6, or a tapered edge. The serrations 104 can be positioned along outer edges of the half-channels 86, 88 in order to engage the biological structure 76 (such as the epineurium of a nerve) and hold it in place and off tension during PTB. The tapered edge (not shown) can include a funnel shape configured to catch the edge of the biological structure 76, on its outer surface, atraumatically, and hold the respective end 78, 80 in place.

Once the biological structure 76 is stabilized, that is, positioned within the first and second half-channels 86, 88 of the first panel 62, the second panel 64 can be closed over the first panel 62 and the device 60 clamped together. More specifically, the second hinge portions 90B can engage the first hinge portions 90A to couple the panels 62, 64 together in a hinged manner. In some aspects, the first hinge portion 90A can be a hook and the second hinge portion 90B can be a catch configured to receive the hook and form the hinge 66. However, other hinge configurations may be realized in some aspects. Additionally, each panel 62, 64 can include a single hinge portion 90A, 90B, two hinge portions 90A, 90B (as shown in FIGS. 4 and 5), or more than two hinge portions 90A, 90B. Furthermore, the hinge 66 shown in FIGS. 4 and 5 can be a releasable hinge 66 such that the two panels 62, 64 can be completed separated from each other. However, in some aspects, the hinge 66 may be a fixed hinge in that the panels 62, 64 remain coupled to each other.

Once the hinge portions 90A, 90B are engaged, the second panel 64 can be rotated, via an axis defined by the hinge 66, or otherwise placed over and aligned with the first panel 62 so that the features of each panel 62, 64 (e.g., at least the cutout 84, the first half-channel 86, and the second half-channel 88) are aligned. As a result, as shown in FIG. 7, the aligned cutouts 84 form the window 74, the aligned first half-channels 86 form the first channel 70, and the aligned second half-channels 88 form the second channel 72. Furthermore, in some applications, the hinge 66 and more specifically, the hinge portions 90A, 90B may be replaced with respective fasteners such that the panels 62, 64 may be aligned and coupled together, though not rotatable relative to a fixed hinge axis.

The panels 62, 64 can further be clamped together via the hemostat 68. While the hemostat 68 may engage the bases 82 along any portion of the device 60, the clamping surfaces 96 can serve as a visible indicator to receive the hemostat 68 and guide proper clamping. For example, in some aspects, as shown in FIGS. 4 and 5, the hinge portions 90A, 90B can be positioned along a first end 106 of each base 82 while the clamping surfaces 96 can be positioned along an opposite second end 108 of the bases 82. In some aspects, the clamping surfaces 96 can be a colored or patterned section on the bases 82 to provide a visual indication to a user. In further aspects the clamping surfaces 96 can have a different surface roughness than the rest of the bases 82 to provide a visual as well as tactile indication to a user, and better engage the hemostat 68.

Accordingly, FIG. 7 illustrates the device 60 in a clamped or assembled state, with two nerve ends 78, 80 ready for repair and positioned in the first and second channels 70, 72, touching end to end within the window 74. That is, the channels 70, 72 provide a place to position and stabilize the nerve ends 78, 80 and have them directly appose one another within with window 74, as well as prevent their retraction to facilitate repair and good tissue apposition.

With further reference to the window 74, as described above, the window 74 is defined by the cutouts 84 in each panel 62, 64. As such, the window 74 is completely open, e.g., devoid of material, providing open access to the apposed nerve ends 78, 80 via the front and the back of the device 60. Accordingly, the window 74 can allow for application of a wrap 110 directly in contact with the joined nerve ends 78, 80 and for application of light to the front and back of the repair. Thus, in some aspects, the window 74 may be considered an irradiation chamber.

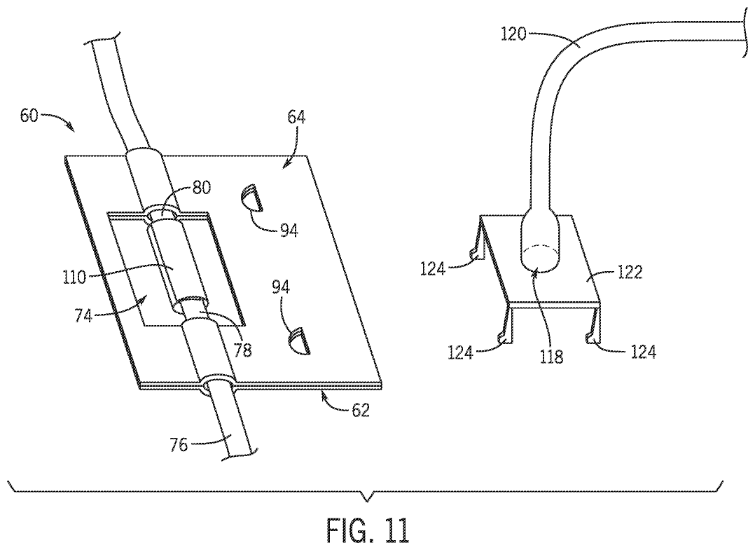

For example, FIG. 8 illustrates the device 60 in the clamped state holding the two nerve ends 78, 80, with a background material 112 placed in the window 74 under the joined nerve ends 78, 80. The background material 112 can be, but is not limited to, paper or a rubber microsurgical background and can provide a setting for placing and supporting the wrap 110 within the window 74 under the joined nerve ends 78, 80. However, in some applications, the wrap 110 can be placed without the background material 112. The wrap 110 can be, but is not limited to, amnion, amnion/chorion, an equivalently thin material containing collagen, or another biomaterial membrane, dyed with a photoactive dye (such as Rose Bengal) in preparation for photochemical bonding.

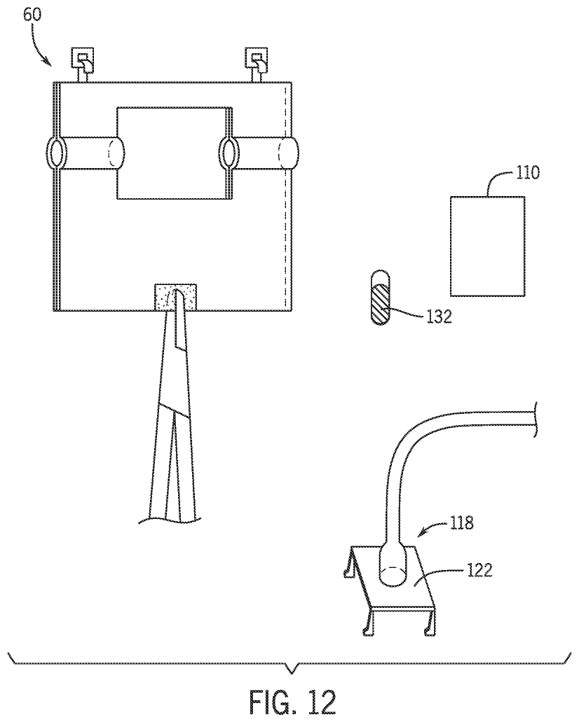

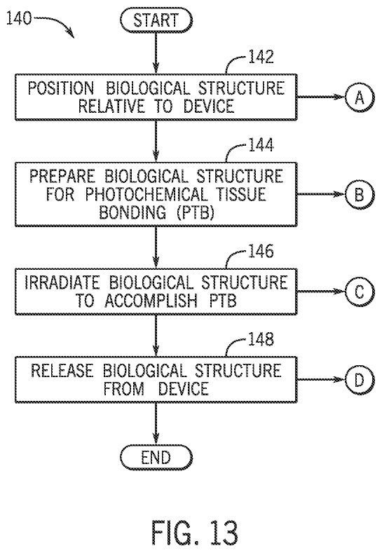

FIG. 9 illustrates a first part of a wrapping procedure, wherein a first end 114 of the wrap 110 is wrapped over the repair site to cover an exposed surface of the joined nerve ends 78, 80. And FIG. 10 illustrates a second part of the wrapping procedure, where a second end 116 of the wrap 110 is wrapped over the first end 114. For example, in some applications, the wrap 110 can be wrapped circumferentially over the joined nerve ends 78, 80 with about 25% overlap. However, another amount of overlap may be used in some applications. Furthermore, while FIGS. 9 and 10 illustrate a “first part” and a “second part” of the wrapping procedure, in some aspects, these parts may be reversed such that the second end 116 is first wrapped over the exposed surface, and the first end 114 is circumferentially wrapped over the second end 116. Once wrapped circumferentially around the nerve ends 78, 80, the wrap 110 can be fixed in place via tissue adhesion or can be directly fixed, e.g., with small hemostatic clips (not shown) along proximal and distal sides of the wrap 110 to hold it in place in close apposition to the nerve surface. Additionally, in some applications, once the wrap 110 is applied, the background material 112 may be removed.

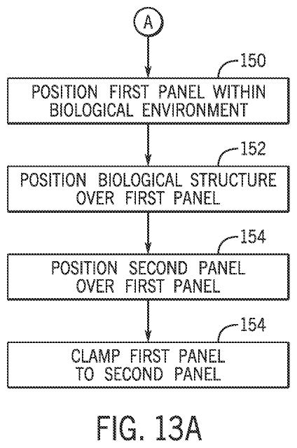

FIG. 11 illustrates the device 60 in the clamped state, holding the nerve ends 78, 80 in place, with the wrap 110 circumferentially wrapped around the nerve ends and ready for irradiation. For example, a light source 118 may be positioned over the window 74 to directly irradiate the wrap 110, activating the photoactive dye to accomplish photochemical bonding. In some aspects, the light source 118 can be, but is not limited to, a laser or light emitting diode (LED) and can emit light at a wavelength specific to the type of photoactive dye used (e.g., 532-nanometer green light for Rose Bengal). Furthermore, in some aspects, as shown in FIG. 11, the light source 118 can be brought to the device 60 via a fiberoptic conduit or cable 120, which can be held by a stand 122 having legs 124 for positioning over the window 74. Furthermore, the legs 124 can be configured to engage or fit into the shoes 94 of the device 60, thus permitting the light source 118 to be coupled to the device 60 and fixed in place over the window 74 for irradiation.

Accordingly, the shoes 94 and the stand 122, as well as the area (or at least side-to-side length) of the window 74 can ensure proper alignment of the light source 118 with the joined nerve ends 78, 80 within the window 74. For example, the shoes 94 and the stand 122 can ensure proper and standardized alignment of the light source 118 over the joined nerve ends 78, 80 to apply light over a fixed length along the biological structure 76 such as, but not limited to, about a 1-centimeter (cm) length, about a 1.5-cm length, about a 2-cm length, or another length. For larger nerve applications, the device 60 can be configured with a larger window 74 (e.g., at least a larger side-to-side window length) to permit a larger wrap application and/or a larger fixed length for irradiation. Furthermore, in some aspects, some portions of the device 10 may be transparent and/or some portions may be reflective in order to allow for uniform exposure of the dyed wrap 110 and nerve ends 78, 80 to light to facilitate bonding.

Furthermore, as noted above, both panels 62, 64 can include the shoes 94, which may be holes or raised openings in the bases 82 sized to receive the legs 124. As a result, the device 60 and the biological structure 76 can be rotated 180 degrees to provide uniform exposure of the wrap 110 and nerve ends 78, 80 to the light from the light source 118 to circumferentially facilitate photochemical bonding. More specifically, the nerve ends 78, 80 can be irradiated via the light source 118 positioned adjacent the first panel 62 (via the stand 122 engaging the shoes 94 on the first panel 62), and then the device 60 can be rotated 180 degrees, causing 180-degree rotation of the nerve as well, and the nerve ends 78, 80 can be irradiated via the light source 118 positioned adjacent the second panel 64 (via the stand 122 engaging the shoes 94 on the second panel 64). Given the pliable nature of most biological structures 76, such as peripheral nerves and vessels, rotation of such structures around their central axes via rotating the device 60 is feasible without causing harm to the structures. Furthermore, as shown in FIG. 11, the device 60 in the clamped state can have a low profile (e.g., both in terms of thickness as well as overall width and/or area) to allow for placement of the device 60 in relatively confined spaces as well as rotation of the device 60 in such spaces. Accordingly, the relative flat profile of the device 60, and the fact that the active area for PTB is confined to the window 74, the device 60 may be suitable for repair of peripheral nerves and vessels in a variety of surgical fields and exposures. Additionally, the hemostat 68 can provide a “handle” for a user to rotate the device 60 when in the clamped state.

It should be noted that in some applications, the device 60 may not include the shoes 94 and the light source 118, either free-standing or via the stand 122, can be manually or otherwise positioned over the window 74 to irradiate the nerve ends 78, 80.

Following successful bonding, the biological structure 76 can be released from the device 60 by unclamping the hemostat 68 and rotating the second panel 64 away from the first panel 62 along an axis defined by the hinge 66, and/or detaching the second panel 64 from the first panel 62. If sutures were used for stabilization, e.g., via the suture holes 92 in the first panel 62, the sutures may be cut. If serrated sides 104 were used for stabilization within the half-channels 86, 88, gentle traction toward the joined ends 78, 80 can release the biological structure 76 from the serrations 104.

Accordingly, the device 60 of some aspects can be used for PTB of a biomaterial membrane 110 to living tissue of a biological structure 76. While FIGS. 4-11 illustrate the device 60 for use with nerve repair, that is, for joining two nerve ends 78, 80 together via a wrap 110, the device 60 may also be used for sealing an intact outer nerve surface. For example, an intact outer nerve surface (e.g., the epineurium and/or the perineurium outer layers of connective tissue) may be injured or compromised from surgical dissection during neurolysis and/or surgical nerve exploration. In such applications, the nerve, which is in continuity (e.g., in contrast to two separate nerve ends 78, 80 described above) can be positioned within both half-channels 86, 88 so that the injured surface is positioned within the cutout 84 and, more specifically, within the window 74 when the device 60 is placed in the clamped state. The nerve surface can then be wrapped with the wrap 110 and irradiated, as discussed above, to seal the epineurium.

Additionally, the device 60 may also be used for sealing other biological structures 76 without a biological membrane 110, that is, for PTB to directly bond two biological structures 76 together. For example, the device 60 may be used for vessel repair, i.e., to join two vessel ends together. In such applications, each vessel end is positioned within a respective half-channel 86, 88, and the ends are telescoped over one another within the cutout 84, for example, over an indwelling bioglass dissolving stent. Each vessel end is also dyed with a photoactive dye. The joined vessel ends can then be irradiated while stabilized within the device 60, as discussed above.

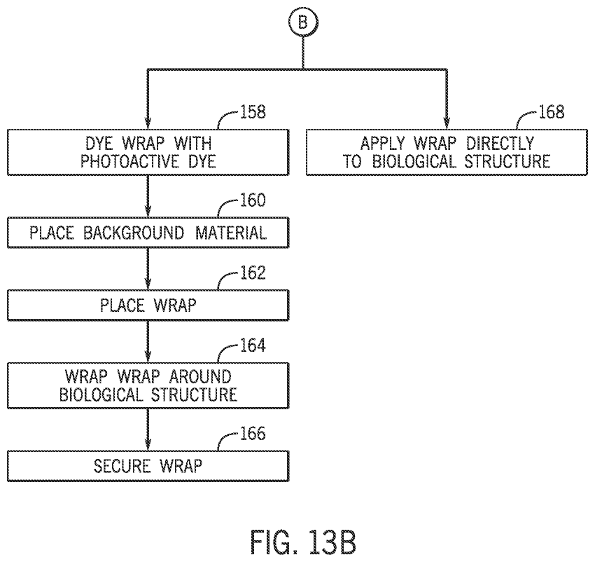

In light of the above, FIG. 12 illustrates a kit 130 according to some aspects of the disclosure. The kit 130 may include a device 60, as described above, a photoactive dye 132 (e.g., stored in a vial or ampule), one or more wraps 110, a fiberoptic cable 120, one or more mounting clips (not shown), and/or a light source 118 (such as a laser or LED). In some applications, the kit 130 may only include single-use items, such as the device 60, the photoactive dye 132, and the wrap 110, while the other items (e.g., the cable 120, the mounting clips, and/or the light source 118) may be available separately for repeat uses with multiple disposable kits 130. Furthermore, in some applications, the wrap 110 may be pre-dyed with the photoactive dye 132, or may be available separately from the kit 130.

In some applications, the kit 130 can include a single device 60 configured to accommodate one biological structure size (e.g., including a single first channel 70 and a single second channel 72 sized to accommodate biological structures 76 having a first range of diameters), thus enabling the kit 130 to be used for one range of biological structure sizes. In some applications, the kit 130 can include multiple devices 60, each configured to accommodate one type of biological structure size, thus enabling the kit to be used for multiple different ranges of biological structure sizes. In some applications, the kit 130 can include a single device 60 configured to accommodate multiple types of biological structure sizes (e.g., including a multiple first channels 70 and a multiple second channels 72, each sized to accommodate biological structures 76 having different ranges of diameters), thus enabling the kit 130 to be used for multiple different ranges of biological structure sizes.

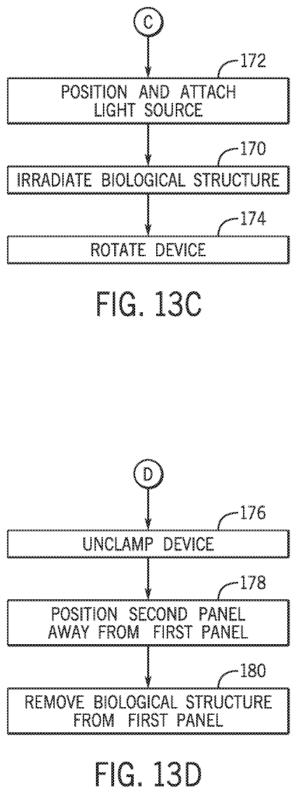

In light of the above, FIG. 13 illustrates a method 140 according to some aspects of the disclosure. Generally, the method 140 may include PTB process for bonding a biological structure. More specifically, the method 140 includes positioning the biological structure 76 relative to the device 60 (step 142), preparing the biological structure 76 for PTB (step 144), irradiating the biological structure 76 to accomplish PTB (step 146), and releasing the biological structure 76 from the device 60 (step 148).

For example, step 142 includes positioning the biological structure 76 relative to the device 60. In some applications, with reference to FIG. 13A, step 142 can include positioning a first panel 62 of the device 60 within a biological environment adjacent the biological structure 76 to be repaired (step 150), positioning the biological structure 76 over the first panel 62 (step 152), positioning the second panel 64 over the first panel 62 (step 154), and clamping the first panel 62 to the second panel 64 (step 156).

At step 152, the biological structure 76 can be positioned over the first panel 62 so that structure 76 is positioned within the first half-channel 86 and the second half-channel 88 and a portion of the structure 76 (e.g., the two ends 78, 80 to be joined, or an injured portion of a continuous structure 76) is positioned within the cutout 84. More specifically, a first portion of the structure 76 (such as the first end 78) can be positioned with the first half-channel 86 and a second portion of the structure 76 (such as the second end 80) can be positioned within the second half-channel 88 such that a third portion of the structure 76 is exposed within the cutout 84. Furthermore, at step 152, the biological structure 76 can be stabilized within the half-channels 86, 88 via sutures or moving the biological structure 76 through the half-channels 86, 88 relative to serrations 104 or other features, as described above. Step 154 can include engaging the first and second hinge portions 90A, 90B of the first and second panels 62, 64, respectively, to form the hinge 66, and rotating the second panel 64 over the first panel 62 along an axis formed by the hinge 66 so that it overlaps and aligns with the first panel 62, thus defining the first channel 70 and the second channel 72 through which the first and second portions of the biological structure 76 are routed, and the window 74 into which the third portion of the biological structure 76 to be bonded is located and exposed. At step 156, the first panel 62 and the second panel 64 are clamped together via the hemostat 68 (e.g., engaged at the clamping surfaces 96 of the panels 62, 64), or otherwise coupled together.

Referring back to the method 140 of FIG. 13, step 144 includes preparing the biological structure 76 for PTB. In some applications, with reference to FIG. 13B, step 144 can include dying or staining the wrap 110 with a photoactive dye (step 158), optionally placing the background material 112 in the window 74 beneath the portion of the biological structure 76 to be bonded (step 160), placing the wrap 110 over the background material 112 and beneath the portion of the biological structure 76 to be bonded (step 162) wrapping the wrap 110 circumferentially around the portion of the biological structure (step 164), and securing the wrap 110 in a wrapped state, e.g., via tissue adhesion or separate clamps (step 166). It should be noted that not all steps 158-166 may be required in some applications. For example, in some applications, the wrap 110 may be pre-dyed, eliminating the need for step 158. As another example, in some applications, the background material 112 may be eliminated, eliminating the need for step 160.

Alternatively or in addition, still referring to FIG. 13B, step 144 can include applying a photoactive dye directly to the biological structure 76 (step 168). For example, step 168 can include applying, spraying, painting, or otherwise adding the photoactive dye to the biological structure 76 via, for example, a cotton tip applicator, sponge, brush, or other applicator.

In some applications, the photoactive dye (also considered a photochemical agent or photosensitizer) can be Rose Bengal, as described above. For example, in one specific application, the photoactive dye can be 0.1% Rose Bengal in a saline solution. However, in other applications, the photoactive dye may be selected from the group consisting of xanthenes, flavins, thiazines, porphyrins, expanded porphyrins, chlorophylls, phenothiazines, cyanines, mono azo dyes, azine mono azo dyes, rhodamine dyes, benzophenoxazine dyes, oxazines, and anthroquinone dyes. In yet other applications, the photochemical agent may selected from the group consisting of Rose Bengal, erythrosine, riboflavin, methylene blue (“MB”), Toluidine Blue, Methyl Red, Janus Green B, Rhodamine B base, Nile Blue A, Nile Red, Celestine Blue, Remazol Brilliant Blue R, riboflavin-5-phosphate (“R-5-P”), N-hydroxypyridine-2-(I H)-thione (“N-HTP”) and photoactive derivatives thereof.

Referring back to the method 140 of FIG. 13, step 146 includes irradiating the biological structure 76 to accomplish PTB. More specifically, with reference to FIG. 13C, step 143 can include irradiating the biological structure, using a light source 118 that emits light at a wavelength configured to cause photoactivation of the dye, for a time period (step 170). In some applications, step 146 can also include first attaching the light source 118 to the device 60, e.g., via the stand 122 and the shoes 94, in order to provide the light source 118 at a standard distance from the biological structure 76 to accomplish irradiation along a predetermined length of the biological structure 76 (step 172). However, in other applications, step 172 may be eliminated and the light source 118 may be manually or otherwise held over the biological structure 76 to be irradiated. Furthermore, in some applications, step 146 can include rotating the device 60 180 degrees (step 174) and repeating steps 170 and, in some applications, step 172 on the back of the biological structure 76 (i.e., where steps 170 and 172 were originally performed on the front of the biological structure 76).

Referring back to the method 140 of FIG. 13, step 148 includes releasing the bonded biological structure 76 from the device 60. For example, with reference to FIG. 13D, step 148 can include unclamping the device 60 (step 176), rotating the second panel 64 away from the first panel 62 and/or removing the second panel 64 from the first panel 62 (step 178), and removing the biological structure 76 from the first half-channel 86 and the second half-channel 88 of the first panel 62, as described above (step 180).

It should be noted that, while the method 140 includes steps that are described above and illustrated in a specific order, in some applications, the method 140 may include certain steps executed in a different order, may not include certain steps described above, and/or may include additional steps not specifically described herein. For example, in some applications, the panels 62, 64 may not be removable from each other and, thus, steps 154 and 178 may include positioning the first panel 62 relative to and away from the second panel 64, respectively, but not fully attaching or detaching the panels 62, 64 to/from each other. Additionally, in some applications, the panels 62, 64 may not be hingedly connected and, thus, steps 154 and 178 may include positioning the first panel 62 relative to and away from the second panel 64, respectively, but not rotating the panels 62, 64 relative to each other.

As another example, in some applications, once the biological structure 76 is removed from the device 60 at step 180, the device 60, any remaining dye, and/or any other single-use components of the kit may be disposed of. Alternatively, in some applications, once the biological structure 76 is removed from the device 60 at step 180, the device 60 and/or any other multi-use components may be sterilized or otherwise cleaned to be used with another biological structure.

In light of the above, aspects of the disclosure provide a device, kit, and/or method to facilitate the process of photochemical tissue bonding of a nerve, or other biological structure, in clinical practice by stabilizing the nerve ends and stabilizing any wrap material around the nerve to maximize the bonding area and facilitate the irradiation of the tissues which have been dyed with a photoactive dye. The device can be an ultrathin, hinged or two part clamp which attaches to the nerve ends to hold them in place and hold the wrap in place. The device further includes an attachment for a light source to then efficiently irradiate the dyed wrap and nerve ends in the wrapped area. The thin or low profile of the device allows for its placement into relatively confined spaces, such as for the purpose of nerve repair and/or nerve sealing.

The present invention has been described in terms of one or more preferred embodiments, and it should be appreciated that many equivalents, alternatives, variations, and modifications, aside from those expressly stated, are possible and within the scope of the invention. Furthermore, the term “about” as used herein means a range of plus or minus 20% with respect to the specified value, more preferably plus or minus 10%, even more preferably plus or minus 5%, most preferably plus or minus 2%. In the alternative, as known in the art, the term “about” indicates a deviation, from the specified value, that is equal to half of a minimum increment of a measure available during the process of measurement of such value with a given measurement tool.

1. A device for performing photochemical tissue bonding on a biological structure, the device comprising:
a first channel sized to receive a first portion of the biological structure;
a second channel sized to receive a second portion of the biological structure; and
an open window positioned between the first channel and the second channel, wherein a third portion of the biological structure is exposed within the open window when the first channel receives the first portion and the second channel receives the second portion so that photochemical tissue bonding may be applied to the third portion.

2. The device of claim 1, further comprising a first panel and a second panel.

3. The device of claim 2, wherein the first panel and the second panel are coupled together and rotatable relative to each other via a hinge.

4. The device of claim 3, wherein each of the first panel and the second panel includes:
a base,
a cutout extending through the base,
a first half-channel in the base, and
a second half-channel in the base.

5. The device of claim 4, wherein, when the second panel is positioned over the first panel, the first half-channel of each of the first panel and the second panel define the first channel, the second half-channel of each of the first panel and the second panel define the second channel, and the cutout of each of the first panel and the second panel define the window.

6. The device of claim 4 and further comprising a clamping tool configured to clamp the second panel to the first panel when the second panel is positioned over the first panel.

7. The device of claim 4, wherein the base of the first panel and the second panel includes suture holes extending therethrough adjacent the first half-channel and the second half-channel.

8. The device of claim 4, wherein the base of the first panel and the second panel includes an attachment site configured to engage a stand for a light source.

9. A kit for performing photochemical tissue bonding on a biological structure, the kit comprising:
a device comprising:
a first channel sized to receive a first portion of the biological structure,
a second channel sized to receive a second portion of the biological structure, and
an open window positioned between the first channel and the second channel, wherein a third portion of the biological structure is exposed within the open window when the first channel receives the first portion and the second channel receives the second portion so that photochemical tissue bonding may be applied to the third portion; and
a photoactive dye.

10. The kit of claim 9 and further comprising a light source.

11. The kit of claim 10 and further comprising a stand configured to hold the light source, wherein the device includes attachment sites configured to engage the stand to position the light source over the window.

12. The kit of claim 9 and further comprising a wrap configured to wrap around the third portion of the biological structure in the window.

13. The kit of claim 12, wherein the wrap is selected from a list comprising: amnion, amnion/chorion, a material containing collagen, and a biomaterial membrane.

14. The kit of claim 12 and further comprising a background material configured to be positioned adjacent the third portion of the biological structure in the window to support the wrap.

15. A method for applying photochemical tissue bonding to a biological structure using a device comprising a first channel, a second channel, and an open window, the method comprising:
positioning the biological structure relative to the device, including positioning a portion of the biological structure in the open window;
preparing the biological structure for photochemical tissue bonding;
irradiating the portion of the biological structure within the window to accomplish photochemical tissue bonding; and
releasing the biological structure from the device.

16. The method of claim 15, wherein positioning the biological structure includes:
positioning the biological structure over a first panel of the device,
positioning a second panel of the device over the first panel to define the first channel, the second channel, and the open window, and
coupling the second panel to the first panel.

17. The method of claim 15, wherein preparing the biological structure for photochemical tissue bonding includes circumferentially wrapping a wrap around the portion of the biological structure, the wrap being stained with a photoactive dye.

18. The method of claim 15, wherein preparing the biological structure for photochemical tissue bonding includes applying a photoactive dye to the portion of the biological structure.

19. The method of claim 18, wherein irradiating the portion of the biological structure includes irradiating with a light source having a wavelength configured to activate the photoactive dye.

20. The method of claim 19, wherein irradiating the portion of the biological structure further includes:
irradiating a front of the portion,
rotating the device, and
irradiating a back of the portion.
